(19) United States
(12) Patent Application Publication
(10) Pub. No.: US 2023/0173516 A1
(43) Pub. Date: Jun. 8, 2023

(54) HAND PIECE FOR AESTHETIC AND MEDICAL DEVICE

(71) Applicant: Soo Chang SON, Yangsan-si (KR)

(72) Inventor: Soo Chang SON, Yangsan-si (KR)

(21) Appl. No.: 17/542,567

(22) Filed: Dec. 6, 2021

(51) Int. Cl. B05B 7/24; B05B 7/04; A61M 11/02; A45D 34/04

(52) U.S. Cl. CPC B05B 7/2491; B05B 7/0475; A61M 11/02; A45D 34/04; A45D 2200/057; A45D 2034/005

(57) ABSTRACT

Disclosed is a hand piece for an aesthetic and medical device including a first body portion having a cylindrical structure in which a cartridge mounting space a second body portion bound to a lower side of the first body portion and having a cylindrical structure having an upper side on which an air pressure providing portion configured to provide air pressure to the cartridge-mounting space and the auxiliary air flow paths is mounted and a spray head portion bound to an upper body of the first body portion and having a central part on which a spray needle bound to an upper body of the cartridge to upwardly spray the liquid cosmetic product or liquid medication accommodated in the cartridge is mounted.

### BACKGROUND

### 1. Field of the Invention

The present invention relates to a hand piece for an aesthetic and medical device, and more particularly, to a hand piece for an aesthetic and medical device which is configured to spray a liquid cosmetic product or liquid medication over the skin of a human body.

### 2. Discussion of Related Art

WO 2009/047173 A2 discloses an atomizer for atomizing a fluid. As shown in FIG. 1, the atomizer according to a related art includes an insertable cartridge configured to store the fluid, a housing configured to accommodate the cartridge, and a pressure generator including a driving spring configured to pressurize a dosage of the fluid to be atomized. The atomizer further includes a transfer tube insertable into the cartridge and collects the dose of fluid from the cartridge when the driving spring is tensed.

WO2009/103510 A1 discloses an atomizer including a pre-inserted cartridge storing a fluid to be atomized. The cartridge includes a flexible transfer tube configured to transfer the fluid from the cartridge.

WO2010/094305 A1 discloses an atomizer for dispensing a liquid. The atomizer includes a cartridge including a liquid for pushing out a liquid from a storage portion using a valve to dispense the liquid and the storage portion accommodating a pressurizing gas.

WO2012/130757 A1 relates to a hand-held device, and more particularly, to an atomizer for discharging a liquid pharmaceutical preparation from a container having a container cap. The liquid pharmaceutical preparation may be based on, for example, an alcoholic solvent. The atomizer having an inserted container includes a sealing system including two sealing portions between the container and a device for preventing a liquid loss, dispersion, and leakage, and gas exchange at this part.

According to a spatial direction of the atomizer/cartridge, there may be an influence on collection of the fluid. Particularly, an appropriate amount of fluid from the cartridge may not be collected, and/or gases or air may be absorbed, and/or a residual amount of fluid may be present in the cartridge after a final dose is completely transferred because an overall volume is not used.

To remedy such problems, WO98/46522 A1 discloses a cartridge including a fluid and a storage medium such as a sponge. A hard or flexible tube for collecting the fluid is firmly attached to a wall of the cartridge or ends at the storage medium capable of freely swing with the collecting tube. The storage medium uniformly maintains a particular amount of fluid even when an end of the tube is above a fluid level of the cartridge. In this way, the fluid may be basically collected from the cartridge fundamentally regardless of spatial orientation. Nevertheless, a particular residual amount of fluid is left in the cartridge or the storage medium from which the fluid is not collectable.

Accordingly, it is necessary to remedy the above problems according to the related arts.

### RELATED ART DOCUMENT

### Patent Document

WO/2009/047173 (published on Apr. 16, 2009)

### SUMMARY OF THE INVENTION

The present invention is directed to providing a hand piece for an aesthetic and medical device which is configured to easily mount a cartridge in which a liquid cosmetic product or liquid medication is accommodated, to stably spray the liquid cosmetic product or liquid medication on a desired portion of skin of a human body, to stably spray an entirety of the liquid cosmetic product or liquid medication hanging on an end of a spray needle, and furthermore, to prevent the liquid cosmetic product or liquid medication from hanging and remaining on the end of the spray needle.

According to an aspect of the present invention, there is provided a hand piece for an aesthetic and medical device including a first body portion having a cylindrical structure in which a cartridge mounting space, in which a cartridge accommodating a liquid cosmetic product or liquid medication is mounted, extends to a certain length in a vertical direction and one or more auxiliary air flow paths are formed to be adjacent to an outer circumferential surface of the cartridge mounting space in a vertical direction, a second body portion bound to a lower side of the first body portion and having a cylindrical structure having an upper side on which an air pressure providing portion configured to provide air pressure to the cartridge-mounting space and the auxiliary air flow paths is mounted, having a lower side on which an air supply line configured to provide compressed air to the air pressure providing portion is mounted, and having a center in which a compressed air flow path, through which the compressed air provided from the air pressure supply line is transferred to the air pressure providing portion, is formed in a center, and a spray head portion bound to an upper body of the first body portion and having a central part on which a spray needle bound to an upper body of the cartridge to upwardly spray the liquid cosmetic product or liquid medication accommodated in the cartridge is mounted.

The first body portion and the second body portion may be bound to each other to form a cylindrical structure grippable by a user's hand.

The first body portion may include a vertically-variable-length structure having a variable length extending in a vertical direction. The vertically-variable-length structure is a structure having a length changed in a sliding manner in a vertical direction by a rotating operation along an outer circumferential surface of the first body portion. A vertical length of the cartridge-mounting space may be changed by the vertically-variable-length structure.

The air pressure providing portion of the second body portion may include a support plate mounted on the upper side of the second body portion in a plate-shaped structure and having a circular plate-shaped structure coming into contact with a lower surface of the first body portion to form a stable binding structure of the first body portion and the second body portion, a central air pressure providing nozzle protruding upward to a certain height from a center of an upper surface of the support plate and inserted with a certain depth into a lower side of the cartridge mounting space to be pressed against and fixed to the lower side, and an auxiliary air pressure providing nozzle formed on one side of the upper surface of the support plate to protrude upward to a certain height and inserted into a lower side of the auxiliary air flow path to a certain depth to be fixedly pressed thereagainst.

In this case, the first body portion may further include a protruding binding and fastening portion protruding downward to a certain length from a lower surface of the first body portion and having a cylindrical structure with an outer circumferential surface along which a binding structure is formed and with an inner circumferential surface formed to have a structure and size corresponding to the outer circumferential surface of the support plate. The second body portion may further include an inwardly-recessed binding and fastening portion inwardly recessed with a certain depth from an upper side of the second body portion, having a binding structure formed along an inner circumferential surface, and having a structure and size corresponding to the protruding binding and fastening portion.

The compressed air flow path of the second body portion may diverge into two or more flow channels, and the flow channels diverging from the compressed air flow path may communicate with a central air pressure providing nozzle and an auxiliary air pressure providing nozzle.

The flow channels diverging from the compressed air flow path may include a first flow channel configured to transfer compressed air to the central air pressure providing nozzle and a second flow channel configured to transfer compressed air to the auxiliary air pressure providing nozzle.

Adjusting valves configured to independently adjust compressed air flow rates may be mounted on the first flow channel and the second flow channel, and an air pressure providing amount control switch configured to independently control operations of the respective adjusting valves may be provided on an outer circumferential surface of the second body portion.

The second body portion may include adjusting valves mounted on the compressed air flow path providing air pressure to the cartridge-mounting space and the auxiliary air flow path to adjust compressed air flow rates and an air pressure providing amount control switch mounted on an outer circumferential surface of the second body portion and configured to control operations of the adjusting valves.

The spray head portion may include a central through hole formed at a position corresponding to an upper end of the spray needle and a side air flow path formed to surround an outer circumferential surface of the spray needle and configured to allow the auxiliary air flow path and a central through hole to communicate with each other.

The compressed air transferred through the compressed air flow path of the second body portion may flow upward along the auxiliary air flow path of the first body portion and may be upwardly spouted outward through the central through hole along the side air flow path of the spray head portion communicating with the auxiliary air flow path.

An auxiliary through hole may be formed on an upper surface of the spray head portion to be spaced at a certain distance apart along an outer circumferential surface of a central through hole in a plan view. The auxiliary through hole may upwardly spout part of the compressed air flowing along a side air flow path of the spray head portion.

The spray head portion may further include a central spray nozzle communicating with the central through hole formed at a position corresponding to an upper end of the spray needle and have a radial spray hole shape spreading upward by a certain angle and an auxiliary spray nozzle communicating with the central through hole near the upper end of the spray needle, spreading upward by a certain angle, and having a donut shape along an outer circumference of the central spray nozzle in a plan view.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a plan view illustrating a cosmetic spraying device according to a related art;

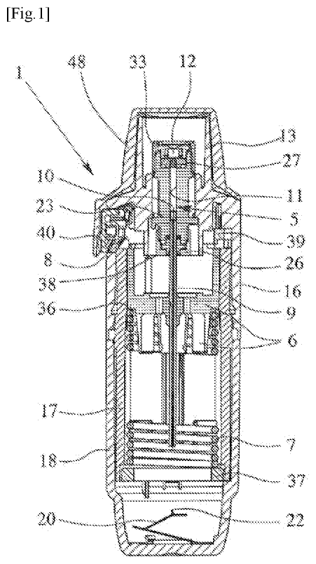

FIG. 2 illustrates a plan view and a front view of a hand piece for an aesthetic and medical device according to one embodiment of the present invention;

FIG. 3 is an exploded longitudinal cross-sectional view of the hand piece of FIG. 2;

FIG. 4 is a cross-sectional view illustrating a state in which a cartridge which accommodates a liquid cosmetic product or liquid medication therein is mounted in a cartridge-mounting space of a first body portion of the hand piece according to one embodiment of the present invention;

FIG. 5 is a cross-sectional view illustrating a state in which a second body portion is mounted below the first body portion of FIG. 4;

FIG. 6 is a cross-sectional view illustrating a state in which a spray head portion is mounted on an upper side of the first body portion of FIG. 5;

FIG. 7 is a cross-sectional view illustrating a state in which the liquid cosmetic product or liquid medication accommodated in the cartridge is sprayed by providing compressed air through an air pressure supply line of the hand piece according to one embodiment of the present invention;

FIG. 8 shows a plan view and a longitudinal cross-sectional view illustrating a spray head portion according to another embodiment of the present invention;

FIG. 9 is a longitudinal cross-sectional view illustrating a central spray nozzle and an auxiliary spray nozzle which are additionally mounted on the spray head portion according to another embodiment of the present invention; and

FIG. 10 is a partial enlarged cross-sectional view illustrating an adjusting valve and an air pressure providing amount control switch which are mounted on a first flow channel and a second flow channel which diverge from a compressed air flow path according to another embodiment of the present invention.

### DETAILED DESCRIPTION OF EXEMPLARY EMBODIMENTS

Hereinafter, exemplary embodiments of the present invention will be described in detail with reference to the attached drawings. Before this, the terms or words used in the specification and the claims should not be limited to general or lexical meanings and should be construed as meanings and concepts coinciding with the technical concept of the present invention.

Throughout the specification, when a member is stated as being located “on” another member, not only the member comes into contact with the other member but also still another member is present therebetween. Throughout the specification, when a portion is stated as “including” a component, unless defined particularly otherwise, it means that the portion may not exclude another component but may further include another component.

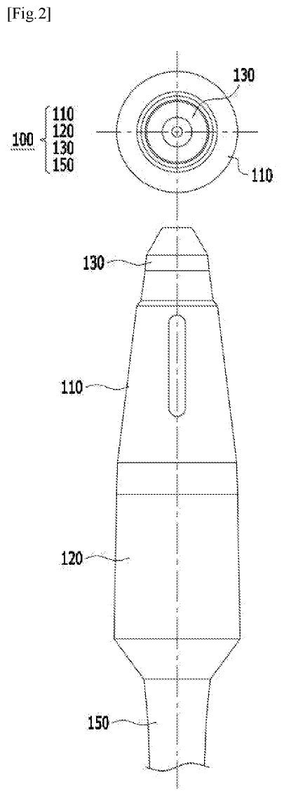

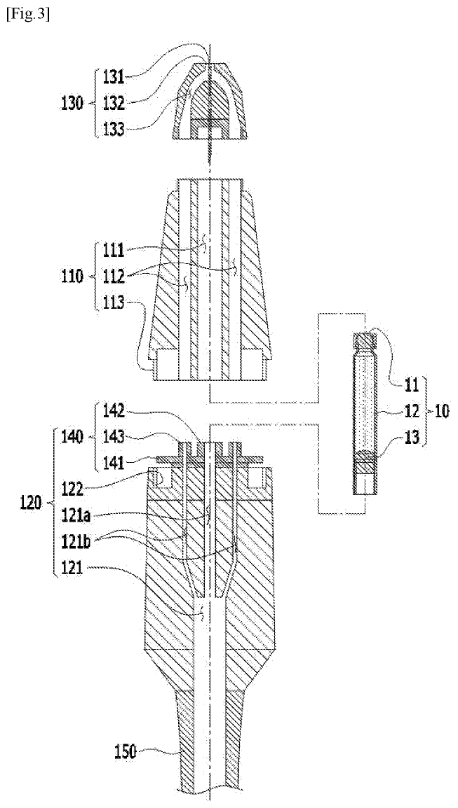

FIG. 2 illustrates a plan view and a front view of a hand piece for an aesthetic and medical device according to one embodiment of the present invention, and FIG. 3 is an exploded longitudinal cross-sectional view of the hand piece of FIG. 2.

Referring to these drawings, a hand piece 100 for an aesthetic and medical device according to the embodiment may include a first body portion 110, a second body portion 120, and a spray head portion 130 of a particular structure so as to easily mount a cartridge in which a liquid cosmetic product or liquid medication is accommodated, to stably spray the liquid cosmetic product or liquid medication on a desired portion of skin of a human body, to stably spray an entirety of the liquid cosmetic product or liquid medication hanging on an end of a spray needle 131, and furthermore, to prevent the liquid cosmetic product or liquid medication from hanging and remaining on the end of the spray needle 131.

Hereinafter, respective components of the hand piece 100 according to the embodiment will be described in detail.

The first body portion 110 according to the embodiment is a component having a cylindrical structure in which a cartridge mounting space 111, in which a cartridge accommodating a liquid cosmetic product or liquid medication is mounted, extends with a certain length in a vertical direction and one or more auxiliary air flow paths 112 are formed to be adjacent to an outer circumferential surface of the cartridge mounting space 111 in a vertical direction.

In some cases, the first body portion 110 may include a vertically-variable-length structure which has a variable length extending in a vertical direction. In this case, the vertically-variable-length structure may be a structure having a vertical length which is slidably changed by a rotating operation along an outer circumferential surface of the first body portion 110. Here, the vertical length of the cartridge mounting space 111 may vary due to the vertically-variable-length structure.

The second body portion 120 is a component bound to a lower side of the first body portion 110. Here, the first body portion 110 and the second body portion 120 may form a cylindrical structure bound to each other to be grippable by a user's hand.

In detail, the first body portion 110 may be a component including a protruding binding having a particular structure and fastening portion 113 and the second body portion 120 may be a component including an inwardly-recessed binding and fastening portion 122 having a particular structure. In more detail, the protruding binding and fastening portion 113 is a component formed to protrude downward with a certain length from a lower surface of the first body portion 110 and may be a cylindrical structure in which a binding structure is formed along an outer circumferential surface and which has an inner circumferential surface formed to have a structure and size corresponding to an outer circumferential surface of a support plate 141. Also, the inwardly-recessed binding and fastening portion 122 is a component formed to be recessed downward with a certain depth from an upper side of the second body portion 120, may have a binding structure formed along an inner circumferential surface, and may be formed to have a structure and size corresponding to the protruding binding and fastening portion 113.

Meanwhile, the cartridge mounting space 111 and an air pressure providing portion 140 configured to provide air pressure to the auxiliary air flow path 112 are mounted on the upper side of the second body portion 120 and an air pressure supply line 150 configured to provide compressed air to the air pressure providing portion 140 are mounted on a lower side of the second body portion 120. The second body portion 120 according to the embodiment has a cylindrical structure in which a compressed air flow path 121, through which the compressed air provided from the air pressure supply line 150 is transferred to the air pressure providing portion 140, is formed in a center.

In some cases, the compressed air flow path 121 of the second body portion 120 may diverge into two or more flow channels. Here, each of the flow channels diverging from the compressed air flow path 121 may communicate with a central air pressure providing nozzle 142 and an auxiliary air pressure providing nozzle 143.

Also, the flow channels diverging from the compressed air flow path 121 may include a first flow channel 121a and a second flow channel 121b. Here, the first flow channel 121a may transfer compressed air to the central air pressure providing nozzle 142, and the second flow channel 121b may transfer compressed air to the auxiliary air pressure providing nozzle 143.

The spray head portion 130 has a structure bound to the upper side of the first body portion 110 and has a central part on which the spray needle 131 bound to an upper side of the cartridge and configured to spray the liquid cosmetic product or liquid medication accommodated in the cartridge upward is mounted.

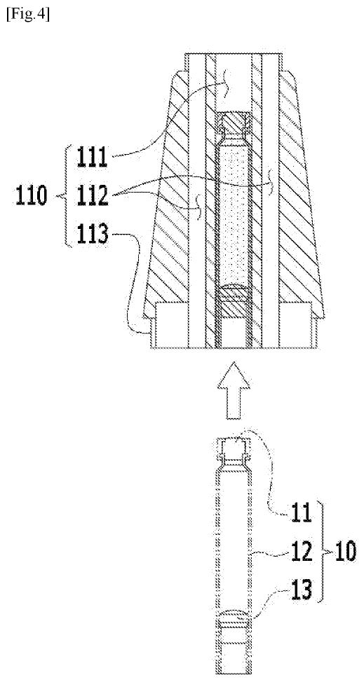

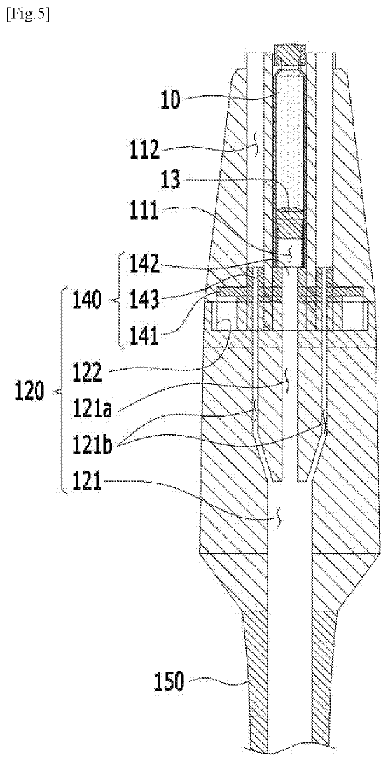

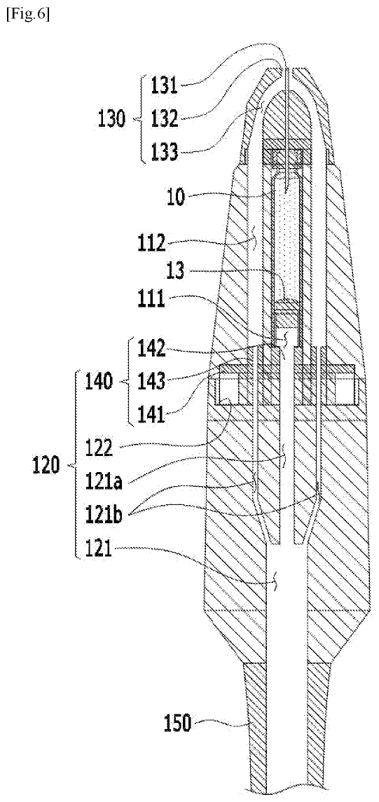

FIG. 4 is a cross-sectional view illustrating a state in which the cartridge which accommodates a liquid cosmetic product or liquid medication therein is mounted in the cartridge-mounting space of the first body portion of the hand piece according to one embodiment of the present invention. FIG. 5 is a cross-sectional view illustrating a state in which the second body portion is mounted below the first body portion of FIG. 4, and FIG. 6 is a cross-sectional view illustrating a state in which the spray head portion is mounted on the upper side of the first body portion of FIG. 5.

Referring to the drawings, the air pressure providing portion 140 of the second body portion 120 according to the embodiment may include the support plate 141, the central air pressure providing nozzle 142, and the auxiliary air pressure providing nozzle 143 of a particular structure.

In detail, the support plate 141 according to the embodiment is a component mounted on the upper side of the second body portion 120 in a plate-shaped structure and may have a circular plate-shaped structure coming into contact with a lower surface of the first body portion 110 to form a stable binding structure of the first body portion 110 and the second body portion 120.

The central air pressure providing nozzle 142 is a component protruding upward to a certain height from a center of an upper surface of the support plate 141 and may be inserted with a certain depth into a lower side of the cartridge mounting space 111 to be pressed against and fixed to the lower side.

The auxiliary air pressure providing nozzle 143 is a component protruding upward to a certain height from one side of an upper surface of the support plate 141 and may be inserted with a certain depth into a lower side of the auxiliary air flow path 112 and pressed against and fixed to the lower side.

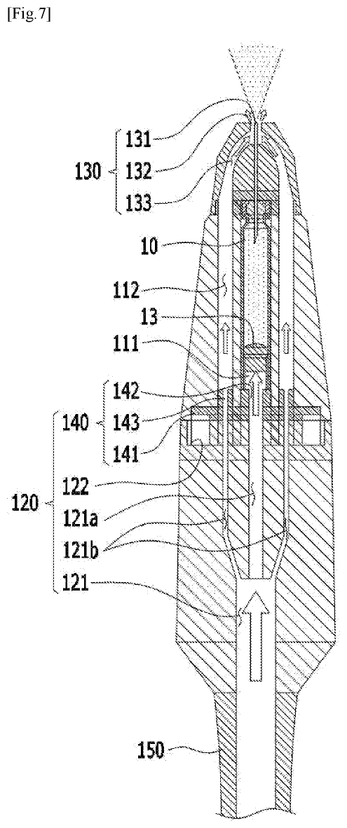

FIG. 7 is a cross-sectional view illustrating a state in which the liquid cosmetic product or liquid medication accommodated in the cartridge is sprayed by providing compressed air through the air pressure supply line of the hand piece according to one embodiment of the present invention.

Referring to FIGS. 2, 6, and 7, the spray head portion 130 according to the embodiment may include a central through hole 132 and a side air flow path 133 of a particular structure.

In detail, the central through hole 132 may be formed at a position corresponding to an upper end of the spray needle 131. Also, the side air flow path 133 is a component formed to surround an outer circumferential surface of the spray needle 131 and may be a structure configured to connect the auxiliary air flow path 112 to the central through hole 132.

Here, as shown in FIG. 7, compressed air transferred through the compressed air flow path 121 of the second body portion 120 may flow upward along the auxiliary air flow path 112 of the first body portion 110 and then may be upwardly spouted outward through the central through hole 132 along the side air flow path 133 of the spray head portion 130 communicating with the auxiliary air flow path 112.

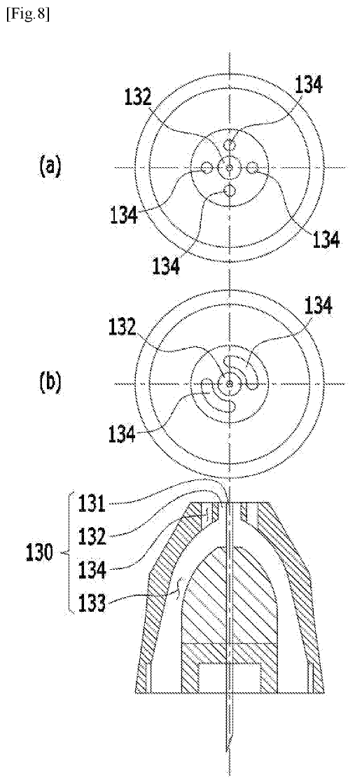

(a) and (b) of FIG. 8 are a plan view and a longitudinal cross-sectional view illustrating a spray head portion according to another embodiment of the present invention.

Referring to (a) and (b) of FIG. 8, an auxiliary through hole 134 disposed to be spaced at a certain distance apart along an outer circumferential surface of the central through hole 132 in a plan view may be formed on an upper surface of the spray head portion 130 according to the embodiment. Here, the auxiliary through hole 134 may upwardly spout part of the compressed air flowing along the side air flow path 133 of the spray head portion 130.

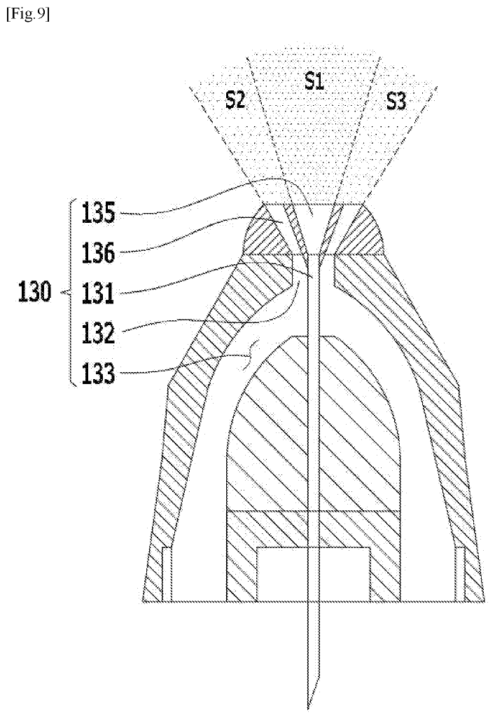

FIG. 9 is a longitudinal cross-sectional view illustrating a central spray nozzle and an auxiliary spray nozzle which are additionally mounted on the spray head portion according to another embodiment of the present invention.

Referring to FIG. 9, the spray head portion 130 according to the embodiment may further include a central spray nozzle 135 and an auxiliary spray nozzle 136 of a particular structure.

In detail, the central spray nozzle 135 may communicate with the central through hole 132 formed at a position corresponding to the upper end of the spray needle 131 and have a radial spray hole shape spreading upward by a certain angle.

Also, the auxiliary spray nozzle 136 may communicate with the central through hole 132 near the upper end of the spray needle 131, may spread upward by a certain angle, and may be a donut shape along an outer circumference of the central spray nozzle 135 in a plan view.

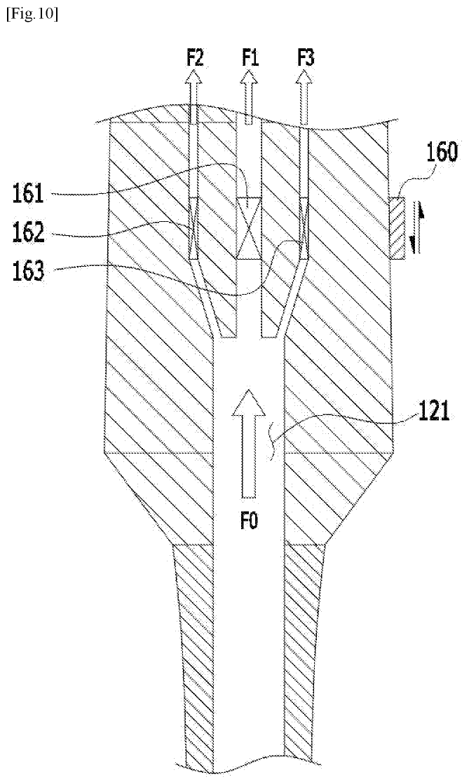

FIG. 10 is a partial enlarged cross-sectional view illustrating an adjusting valve and an air pressure providing amount control switch which are mounted on a first flow channel and a second flow channel which diverge from a compressed air flow path according to another embodiment of the present invention.

Referring to FIGS. 6, 7, and 10, adjusting valves 161, 162, and 163 configured to independently adjust compressed air flow rates may be mounted on the first flow channel 121a and the second flow channel 121b of the hand piece according to the embodiment. Simultaneously, an air pressure providing amount control switch 160 configured to independently control operations of the respective adjusting valves may be mounted on an outer circumferential surface of the second body portion 120.

As described above, according to the present invention, the hand piece for an aesthetic and medical device may include the first body portion 110, the second body portion 120, and the spray head portion 130 of a particular structure so as to easily mount a cartridge in which a liquid cosmetic product or liquid medication is accommodated, to stably spray the liquid cosmetic product or liquid medication on a desired portion of skin of a human body, to stably spray an entirety of the liquid cosmetic product or liquid medication hanging on an end of the spray needle 131, and furthermore, to prevent the liquid cosmetic product or liquid medication from hanging and remaining on the end of the spray needle 131.

As described above, according to the present invention, a hand piece for an aesthetic or medical device may include a first body portion, a second body portion, and a spray head portion of a particular structure so as to easily mount a cartridge in which a liquid cosmetic product or liquid medication is accommodated, to stably spray the liquid cosmetic product or liquid medication on a desired portion of skin of a human body, to stably spray an entirety of the liquid cosmetic product or liquid medication hanging on an end of a spray needle, and furthermore, to prevent the liquid cosmetic product or liquid medication from hanging and remaining on the end of the spray needle.

Only the particular embodiments of the present invention have been described above in the detailed description of the present invention. However, the present invention should be construed as being not limited to the particular form stated above in the detailed description and should be construed as including all modifications, equivalents, and substitutes within the range of the present invention defined by the attached claims.

That is, the present invention is not limited to the particular embodiment and description and a variety of modifications may be made by one of ordinary skill in the art without departing from the concept of the present invention and be within the protective scope of the present invention.

What is claimed is:

1. A hand piece for an aesthetic and medical device, comprising:
a first body portion (110) having a cylindrical structure in which a cartridge mounting space (111), in which a cartridge accommodating a liquid cosmetic product or liquid medication is mounted, extends with a certain length in a vertical direction and one or more auxiliary air flow paths (112) are formed to be adjacent to an outer circumferential surface of the cartridge mounting space (111) in a vertical direction;
a second body portion (120) bound to a lower side of the first body portion (110) and having a cylindrical structure having an upper side on which an air pressure providing portion (140) configured to provide air pressure to the cartridge-mounting space (111) and the auxiliary air flow paths (112) is mounted, having a lower side on which an air supply line (150) configured to provide compressed air to the air pressure providing portion (140) is mounted, and having a center in which a compressed air flow path (121), through which the compressed air provided from the air pressure supply line (150) is transferred to the air pressure providing portion (140), is formed in a center; and
a spray head portion (130) bound to an upper body of the first body portion (110) and having a central part on which a spray needle (131) bound to an upper body of the cartridge to upwardly spray the liquid cosmetic product or liquid medication accommodated in the cartridge is mounted.

2. The hand piece of claim 1, wherein the air pressure providing portion (140) of the second body portion (120) comprises:
a support plate (141) mounted on the upper side of the second body portion (120) in a plate-shaped structure and having a circular plate-shaped structure coming into contact with a lower surface of the first body portion (110) to form a stable binding structure of the first body portion (110) and the second body portion (120);
a central air pressure providing nozzle (142) protruding upward to a certain height from a center of an upper surface of the support plate (141) and inserted with a certain depth into a lower side of the cartridge mounting space (111) to be pressed against and fixed to the lower side; and
an auxiliary air pressure providing nozzle (143) formed on one side of the upper surface of the support plate (141) to protrude upward to a certain height and inserted into a lower side of the auxiliary air flow path (112) to a certain depth to be fixedly pressed thereagainst.

3. The hand piece of claim 1, wherein the second body portion (120) comprises:
adjusting valves (161), (162), (163) mounted on the compressed air flow path (121) providing air pressure to the cartridge-mounting space (111) and the auxiliary air flow path (112) to adjust compressed air flow rates; and
an air pressure providing amount control switch (160) mounted on an outer circumferential surface of the second body portion (120) and configured to control operations of the adjusting valves (161), (162), (163).

4. The hand piece of claim 1, wherein the spray head portion (130) comprises:
a central through hole (132) formed at a position corresponding to an upper end of the spray needle (131); and
a side air flow path (133) formed to surround an outer circumferential surface of the spray needle (131) and configured to allow the auxiliary air flow path (112) and a central through hole (132) to communicate with each other.

5. The hand piece of claim 4, wherein the compressed air transferred through the compressed air flow path (121) of the second body portion (120) flows upward along the auxiliary air flow path (112) of the first body portion 110 and is upwardly spouted outward through the central through hole (132) along the side air flow path (133) of the spray head portion (130) communicating with the auxiliary air flow path (112).
